(19) United States
(12) Patent Application Publication
(10) Pub. No.: US 2026/0263348 A1
(43) Pub. Date: Sep. 10, 2026

(54) COMPOSITION FOR PREVENTING OR IMPROVING SKIN CIRCADIAN RHYTHM DISORDER COMPRISING TEA TREE EXTRACT

(71) Applicant: AMOREPACIFIC CORPORATION, Seoul (KR)

(72) Inventors: Si Young CHO, Yongin-si (KR); Hee Young Jeon, Yongin-si (KR); Kyeong Hwan Hwang, Yongin-si (KR); Yu Mi Kang, Yongin-si (KR); Ji Yong Jung, Yongin-si (KR); Shin Hyoung Kim, Yongin-si (KR); A Young Kim, Yongin-si (KR); Hyoung June Kim, Yongin-si (KR); Yong Deog Hong, Yongin-si (KR)

(73) Assignee: AMOREPACIFIC CORPORATION, Seoul (KR)

(21) Appl. No.: 19/560,472

(22) Filed: Mar. 9, 2026

(30) Foreign Application Priority Data
Mar. 10, 2025 (KR) 10-2025-0030802

(51) Int. Cl. A61K 8/9789; A61K 8/49; A61Q 17/04

(52) U.S. Cl. CPC A61K 8/9789; A61K 8/498; A61Q 17/04; A61K 2800/522; A61K 2800/92

(57) ABSTRACT

The present specification relates to a composition comprising a Jangwon No. 3 tea plant extract as an active ingredient. The composition, by comprising the Jangwon No. 3 tea plant extract as the active ingredient, may provide an effect of improving time dependence of a cell proliferation cycle by normalizing expression of Bmal1, which is a clock gene. Through this, by changing a circadian rhythm in which much damage is caused by ultraviolet irradiation, inherent ultraviolet resistance of cells is enhanced, and side effects such as erythema due to ultraviolet rays can be prevented and improved.

### CROSS REFERENCE TO RELATED APPLICATION

The present application claims priority to Korean Patent Application No. 10-2025-0030802, filed Mar. 10, 2025, the entire contents of which are hereby incorporated by this reference.

### BACKGROUND OF THE INVENTION

### Field of the Invention

The present specification describes a composition comprising a tea plant extract.

### Description of the Related Art

Stress, irregular sleep hours such as shift work, aging, and the like induce expression abnormality of clock genes regulating a circadian rhythm of cells, and by this, various physiological changes are caused. Particularly, it is being reported that expression decrease of Bmal1 protein as a master of the clock genes not only decreases cell proliferation itself but also induces disturbance of a cell proliferation cycle. If expression of Bmal1 is decreased, time dependence of the cell proliferation cycle disappears, and thus DNA synthesis, which mainly occurred at night, also occurs during the day. Since a nuclear envelope which protected DNA disappears when DNA synthesis occurs, if the DNA synthesis occurs during the day, very large damage is generated by ultraviolet rays. Therefore, when received the same ultraviolet irradiation, side effects such as erythema and itching may more occur, but until now, a solution of improving ultraviolet sensitivity of skin itself through such circadian rhythm regulation has not been reported.

### SUMMARY OF THE INVENTION

In one aspect, the present disclosure has been made in an effort to provide a composition capable of improving inherent ultraviolet resistance of cells by regulating clock genes to change a circadian rhythm in which much damage is caused by ultraviolet irradiation.

In one aspect, one embodiment of the present disclosure provides a composition for preventing or improving circadian rhythm disorders of skin, comprising a Jangwon No. 3 tea plant extract as an active ingredient.

The composition according to one embodiment of the present disclosure, by comprising the Jangwon No. 3 tea plant extract among various tea plant varieties as the active ingredient, may provide an effect of improving time dependence of the cell proliferation cycle by normalizing expression of Bmal1, which is a clock gene. That is, according to the present disclosure, by changing the circadian rhythm in which much damage is caused by ultraviolet irradiation, inherent ultraviolet resistance of cells is enhanced, and through this, side effects such as erythema due to ultraviolet rays can be prevented and improved.

### BRIEF DESCRIPTION OF THE DRAWINGS

FIG. 1 is a view showing a result of comparing an expression promotion effect of a clock gene Bmal1 protein of Example 1 (JW3) and Comparative Example 1 (GTE) according to one embodiment of the present disclosure.

FIG. 2 is a view showing a result of comparing a time dependence recovery effect of a cell growth cycle of Example 1 (JW3) and Comparative Example 1 (GTE) according to one embodiment of the present disclosure.

FIG. 3 is a view showing a graph comparing an improvement effect of repairing DNA damaged by ultraviolet rays of Example 1 (JW3) and Comparative Example 1 (GTE) according to one embodiment of the present disclosure.

FIG. 4 is a view showing fluorescence images comparing the improvement effect of repairing DNA damaged by ultraviolet rays of Example 1 (JW3) and Comparative Example 1 (GTE) according to one embodiment of the present disclosure.

FIG. 5 is a view showing a result of comparing an ultraviolet resistance enhancement effect in an artificial epidermis model treated with Example 1 (JW3) and Comparative Example 1 (GTE) according to one embodiment of the present disclosure.

FIG. 6 is a view showing a result of comparing the ultraviolet resistance enhancement effect in the artificial epidermis model treated with Example 1 (JW3) and Comparative Example 1 (GTE) according to one embodiment of the present disclosure using a skin proliferation marker Ki67.

FIG. 7 is a view showing a result of comparing the ultraviolet resistance enhancement effect in the artificial epidermis model treated with Example 1 (JW3) and Comparative Example 1 (GTE) according to one embodiment of the present disclosure using a skin barrier marker filaggrin (FLG).

FIG. 8 is a view showing a result of comparing an inhibitory effect on production of prostaglandin, which is an erythema-inducing substance by ultraviolet rays, of Example 1 (JW3) and Comparative Example 1 (GTE) according to one embodiment of the present disclosure.

### DETAILED DESCRIPTION OF THE INVENTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to include all modifications, equivalents, or substitutions that fall within the scope of the spirit and art of the present disclosure. Singular expressions include plural expressions unless clearly described as different meanings in the context. In the present application, the terms “comprises,” “comprising,” “includes,” “including,” “containing,” “has,” “having” or other variations thereof are inclusive and therefore specify the presence of stated features, integers, steps, operations, constituent elements, components (or parts), or combinations thereof, but do not exclude the presence or addition of one or more other features, integers, steps, operations, constituent elements, components (or parts), or combinations thereof.

One embodiment of the present disclosure may provide a composition for preventing or improving circadian rhythm disorders of skin, comprising a Jangwon No. 3 tea plant extract as an active ingredient.

Another embodiment may provide a use of the Jangwon No. 3 tea plant extract for use in manufacture of a composition for preventing or improving circadian rhythm disorders of skin. Another embodiment may provide a method for preventing or improving circadian rhythm disorders of skin, comprising administering the Jangwon No. 3 tea plant extract in an effective amount to a subject in need thereof. Another embodiment may provide the Jangwon No. 3 tea plant extract as an active ingredient for use in a composition for preventing or improving circadian rhythm disorders of skin. In addition, a non-therapeutic use of the Jangwon No. 3 tea plant extract as an active ingredient for preventing or improving circadian rhythm disorders of skin may be provided.

In the present disclosure, the Jangwon No. 3 is a variety name of a new variety of Camellia sinensis, Theaceae, and a scientific name is Camellia sinensis L. The Jangwon No. 3 exhibits morphological characteristics that young shoots with one bud and three leaves and a length of a leaf blade are on a long side, a posture of the leaf blade is upward, an intensity of a color of the leaf blade is medium, and a position where a flower style splits is high. Compared with Yabukita, which is a variety generally used for a green tea extract, it exhibits morphological differences that a width of a leaf blade is narrow, a shape of the leaf blade is a narrower ellipse, and a serrated shape of a leaf blade edge is deep. In one embodiment, the Jangwon No. 3 may be KCTC (Korean Collection for Type Cultures) 12213BP deposited on May 14, 2012 with the Korea Research Institute of Bioscience and Biotechnology.

Also, the extract of the Jangwon No. 3 tea plant also exhibits differences in types and contents of constituent components from an extract of a general tea plant variety.

In one embodiment, the extract of the Jangwon No. 3 tea plant may be one obtained by processing buds or leaves of Jangwon No. 3 tea plant.

In one embodiment, the extract of the Jangwon No. 3 tea plant may comprise methylated catechin (epigallocatechin-3-O-(3-O-methyl)-gallate (EGCG3″Me)) in a high content of about 10 times or more than the extract of the general tea plant variety. Specifically, the Jangwon No. 3 tea plant extract may comprise the methylated catechin in 0.5 to 10 wt % relative to a total weight of the extract. More specifically, the Jangwon No. 3 tea plant extract may comprise the methylated catechin in 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 6 wt % or more, 7 wt % or more, 8 wt % or more, or 9 wt % or more relative to the total weight of the extract, and may be comprised in 10 wt % or less, 9 wt % or less, 8 wt % or less, 7 wt % or less, 6 wt % or less, 5 wt % or less, 4.5 wt % or less, 4 wt % or less, 3.5 wt % or less, 3 wt % or less, 2.5 wt % or less, 2 wt % or less, 1.5 wt % or less, or 1 wt % or less.

In one embodiment, the extract of the Jangwon No. 3 tea plant may further comprise one or more selected from the group consisting of gallocatechin, epigallocatechin, catechin, epicatechin, epigallocatechin gallate, gallocatechin gallate, and epicatechin gallate in addition to the methylated catechin.

In one embodiment, the extract of the Jangwon No. 3 tea plant may comprise 0.5 to 10 wt %, more specifically, 1 to 3 wt % of methylated catechin relative to the total weight of the extract. In one embodiment, the extract of the Jangwon No. 3 tea plant may comprise 0.41 to 1 wt %, more specifically, 0.45 to 0.7 wt % of gallocatechin relative to the total weight of the extract. In one embodiment, the extract of the Jangwon No. 3 tea plant may comprise 8 to 15 wt %, more specifically, 10 to 15 wt % of epigallocatechin relative to the total weight of the extract. In one embodiment, the extract of the Jangwon No. 3 tea plant may comprise 0.3 to 1 wt %, more specifically, 0.4 to 0.6 wt % of catechin relative to the total weight of the extract. In one embodiment, the extract of the Jangwon No. 3 tea plant may comprise 2.1 to 10 wt %, more specifically, 2.5 to 4.5 wt % of epicatechin relative to the total weight of the extract. In one embodiment, the extract of the Jangwon No. 3 tea plant may comprise 16 to 30 wt %, more specifically, 18 to 20 wt % of epigallocatechin gallate relative to the total weight of the extract. In one embodiment, the extract of the Jangwon No. 3 tea plant may comprise 0.11 to 0.5 wt %, more specifically, 0.15 to 0.4 wt % of gallocatechin gallate relative to the total weight of the extract. In one embodiment, the extract of the Jangwon No. 3 tea plant may comprise 3 to 15 wt %, more specifically, 4 to 10 wt % of epicatechin gallate relative to the total weight of the extract.

In one embodiment, the extract of the Jangwon No. 3 tea plant may be one obtained by processing first-flush tea leaves of Jangwon No. 3 tea plant.

Generally, for tea plant, when harvested once, leaves grow from one bud and one leaf to five leaves, and such a harvesting process is repeatedly performed about four times a year. When the leaves of tea plant are classified according to a harvesting period, these may be named first-flush tea, second-flush tea, third-flush tea, and fourth-flush tea. Specifically, the first-flush tea may be one in which tea leaves are harvested for a first time by May of a corresponding year, the second-flush tea may be one harvested for a second time after harvesting the first-flush tea of the corresponding year (harvested in June to July), the third-flush tea may be one harvested for a third time after harvesting the second-flush tea of the corresponding year (harvested in August), and the fourth-flush tea may be one harvested for a fourth time after harvesting the third-flush tea of the corresponding year (harvested on or after September). Also, when the leaves of tea plant are classified according to leaf growth, they may be divided into a first leaf to a fifth leaf according to an order in which leaves emerge from one tea plant (one bud), and it takes about 30 days to 35 days until becoming the fifth leaf after sprouting.

From the above aspect, the ‘first-flush tea’ according to one embodiment may mean one in which tea leaves are harvested for the first time by May of the corresponding year. The ‘first-flush tea’ according to one embodiment may mean one in which tea leaves are harvested at a one-bud and five-leaf stage. For example, the extract of the Jangwon No. 3 tea plant may be one in which the upper first leaf to third leaf of a new shoot are collected based on a period when an opening degree of the one-bud and five-leaf stage is 70% during a first-flush tea period of the Jangwon No. 3 tea plant. In the present disclosure, general green tea corresponding to the first-flush tea means one using tea leaves collected at remaining periods excluding the first-flush tea.

In one embodiment, the Jangwon No. 3 tea plant extract may be an aqueous ethanol solution extract of Jangwon No. 3 tea plant leaves.

In one embodiment, the Jangwon No. 3 tea plant extract may be one manufactured by an extraction method comprising: a step of steaming the tea leaves by steaming and then drying; a step of pulverizing and powdering a dried product obtained in the step; a step of extracting the powdered tea leaves using an aqueous ethanol solution as an extraction solvent; and a step of filtering an extract obtained in the extraction step and then concentrating under reduced pressure to obtain an extract. In one embodiment, the steaming by steaming may be steaming by passing the tea leaves through a steamer for 20 to 60 seconds. In one embodiment, the concentration under reduced pressure may be performed at 40 to 45° C.

In one embodiment, the composition may comprise the Jangwon No. 3 tea plant extract in 0.0001 to 50 wt % relative to a total weight of the composition. Specifically, the composition may comprise the Jangwon No. 3 tea plant extract in 0.0001 wt % or more, 0.001 wt % or more, 0.01 wt % or more, 0.1 wt % or more, 1 wt % or more, 5 wt % or more, 10 wt % or more, 15 wt % or more, 20 wt % or more, 25 wt % or more, 30 wt % or more, 35 wt % or more, 40 wt % or more, or 45 wt % or more relative to a total weight of the composition, and may comprise the same in 50 wt % or less, 45 wt % or less, 40 wt % or less, 35 wt % or less, 30 wt % or less, 25 wt % or less, 20 wt % or less, 15 wt % or less, 10 wt % or less, 5 wt % or less, 1 wt % or less, or 0.1 wt % or less.

In one embodiment, the circadian rhythm disorders of skin may be generated by biological rhythm disturbance. Also, the Jangwon No. 3 tea plant extract may prevent or improve skin damage due to ultraviolet rays through improvement of the circadian rhythm disorders of skin.

From the above aspect, the composition according to one embodiment may be for preventing or improving skin damage due to ultraviolet rays. Another embodiment may provide a use of the Jangwon No. 3 tea plant extract for use in manufacture of a composition for preventing or improving skin damage due to ultraviolet rays. Another embodiment may provide a method for preventing or improving skin damage due to ultraviolet rays, comprising administering the Jangwon No. 3 tea plant extract in an effective amount to a subject in need thereof. Another embodiment may provide the Jangwon No. 3 tea plant extract as an active ingredient for use in a composition for preventing or improving skin damage due to ultraviolet rays. In addition, a non-therapeutic use of the Jangwon No. 3 tea plant extract as an active ingredient for preventing or improving skin damage due to ultraviolet rays may be provided.

In one embodiment, the composition may be targeted for the 40s or 45 years of age or older in which skin turnover is slow.

In one embodiment, the composition according to one embodiment of the present disclosure may be a cosmetic composition. The cosmetic composition may be formulated by comprising a cosmetologically or dermatologically acceptable medium or base. This refers to all formulations suitable for topical application, and may be for transdermal administration. For example, it may be provided in a form of a solution, a gel, a solid, a paste anhydrous product, an emulsion obtained by dispersing an oil phase in an aqueous phase, a suspension, a microemulsion, microcapsules, microgranules, or ionic (liposome) and non-ionic vesicle dispersants, or in a form of a cream, a toner, a lotion, a powder, an ointment, a spray, or a concealer stick. Also, it may be used in a form of a foam or in a form of an aerosol composition further comprising a compressed propellant. As another example, it may be a formulation of an injectable such as a filler, a patch, and the like, but is not limited thereto. These compositions may be manufactured according to conventional methods in the art.

As one embodiment, the composition according to one embodiment of the present disclosure, in addition to the active ingredient, may comprise other components capable of preferably giving a synergistic effect to a main effect within a range that does not damage the main effect, and in addition to the active ingredient of the present disclosure, other components may be appropriately selected and blended by those skilled in the art without difficulty according to a formulation or a purpose of use of other cosmetic compositions. Also, as one embodiment, the cosmetic composition of the present disclosure, in addition to the above components, may comprise other components usually blended in a cosmetic composition as necessary. For example, a moisturizer, an emollient, organic and inorganic pigments, organic powder, a UV absorber, a preservative, a bactericide, an antioxidant, a plant extract, a pH adjuster, alcohol, colorants, fragrance, a blood circulation promoter, a cooling agent, an antiperspirant, purified water, and the like may be mentioned. Other blending components capable of being comprised in the cosmetic composition of the present disclosure are not limited thereto, and a blending amount of the components is possible within a range that does not damage the purpose and effect of the present disclosure.

The composition according to one embodiment of the present disclosure may be an oral composition comprising the active ingredient. As one embodiment, the composition may be a food composition. For example, it may be processed into functional foods such as fermented milk, cheese, yogurt, juice, probiotic preparations, and health foods comprising the active ingredient, and may be used in a form of various other food additives. As one embodiment, the composition may be a composition for health food. As one embodiment, the composition for health food may be formulated into a pill, a capsule, a tablet, granules, a caramel, or a drink, or the like. As another embodiment, it may also be processed in a form of a liquid, powder, granules, a tablet, or a tea bag, or the like. The composition may be administered by various methods such as simple drinking, injection administration, a spray method, or a squeeze method. The composition may comprise other components and the like capable of giving a synergistic effect to a main effect within a range that does not damage the main effect of the present disclosure. For example, to improve physical properties, additives such as fragrance, a colorant, a bactericide, an antioxidant, a preservative, a moisturizer, a thickener, inorganic salts, an emulsifier, and a synthetic polymer substance may be further comprised. In addition to that, auxiliary components such as water-soluble vitamins, oil-soluble vitamins, high molecular peptides, high molecular polysaccharides, and seaweed extract may be further comprised. The components may be appropriately selected and blended by those skilled in the art according to a formulation or a purpose of use, and an addition amount thereof may be selected within a range that does not damage the purpose and effect of the present disclosure. For example, the addition amount of the components may be 0.0001 wt % to 99.9 wt % based on a total weight of the composition.

Hereinafter, the present disclosure will be described in detail with reference to Examples, Comparative Examples, and Test Examples. It will be obvious to those having ordinary knowledge in the art that these are merely presented illustratively to describe the present disclosure more specifically, and a scope of the present disclosure is not limited by these Examples, Comparative Examples, and Test Examples.

### [Example 1] Manufacture of Jangwon No. 3 Tea Plant Extract

Jangwon No. 3 (Osulloc Farm Co., Ltd., Agricultural Corporation) was collected from the upper first leaf to third leaf of a new shoot based on a period when an opening degree of a one-bud and five-leaf stage was 70% during a first-flush tea (early May) period, and was steamed by passing through a steamer for 40 seconds, dried in a dryer at 80° C., and then powder-processed by a pulverizer, sieved through a 60-mesh sieve, and powdered. To 100 g of the pulverized sample of the Jangwon No. 3 thus manufactured, 1 L of a 70% (v/v) aqueous ethanol solution (anhydrous ethanol 70% (v/v)+distilled water 30% (v/v)) was added, and after performing reflux extraction and filtering, it was concentrated under reduced pressure at 40 to 45° C., and finally 19.2 g of a dried extract of leaves of a new variety of tea plant (Jangwon No. 3) was secured.

### Comparative Example 1

A green tea extract from a general tea plant was manufactured in the same method as in Example 1, except that a variety of tea plant was Yabukita (Samkyung Costech).

### [Test Example 1] Comparison of Catechin Composition Comprised in Tea Plant Extract

For a comparative analysis of catechin components comprised in the tea plant extracts of Example 1 and Comparative Example 1 manufactured above, the following experiment was performed.

After dissolving each of the extracts in 50% methanol to make a 10,000 ppm solution, component analysis (Waters Co., 2695/2996 PDA detector) was performed using high-performance liquid chromatography (HPLC) (Waters Co.). For a stationary phase, a Thermo Syncronis C18 (4.6×250 mm, 5 μm), P/N: 97105-254630 column was used, and for a mobile phase, a composition ratio as shown in Table 1 below was used.

TABLE 1

| Time (minutes) | A: 0.1% Acetic acid in water | B: Acetonitrile |
|---|---|---|
| 0 | 90 | 10 |
| 10.0 | 90 | 10 |
| 30.0 | 85 | 15 |
| 53.0 | 80 | 20 |
| 53.1 | 5 | 95 |
| 55.0 | 5 | 95 |
| 55.1 | 90 | 10 |
| 60.0 | 90 | 10 |

As shown in Table 2 below, it was confirmed that the extract of Jangwon No. 3 leaves comprises epigallocatechin-3-O-(3-O-methyl) gallate (EGCG3″Me; 3″-O-Me-EGCG) in an amount of 2.5 wt % based on a total weight of the extract, which is comprised in a high content of 10 times or more than a general tea plant variety.

TABLE 2

| | Example 1 | Comparative |
|---|---|---|
| | (Jangwon | Example 1 (General |
| Item | No. 3 extract) | green tea extract) |
| (1) gallocatechin (GC) | 0.5 | 0.4 |
| (2) epigallocatechin (EGC) | 12.7 | 7.7 |
| (3) catechin (C) | 0.5 | 0.2 |
| (4) epicatechin (EC) | 3.1 | 2.0 |
| (5) epigallocatechin gallate (EGCG) | 18.8 | 15.1 |
| (6) gallocatechin gallate (GCG) | 0.2 | 0.1 |
| (7) epicatechin gallate (ECG) | 5.3 | 2.6 |
| (8) catechin gallate (CG) | N/D | N/D |
| | (Not Detected) | |
| Total content of 8 types of catechins | 41.1 | 28.1 |
| 3”-O-Me-EGCG content | 2.5 | 0.2 |

(wt %)

### [Test Example 2] Confirmation of Expression Promotion Effect of Clock Gene Bmal1

To compare an expression promotion effect of Bmal1, which is a clock gene, of the extracts of Example 1 and Comparative Example 1, the following experiment was performed.

First, aging-induced skin keratinocytes were prepared. Specifically, skin keratinocytes (normal human epidermal keratinocytes, NBIEK) were seeded to be 12,000 cell s/cm² and cultured until reaching 8000 confluency. To the cell culture, UVB 20 mJ/cm² was repeatedly irradiated once a day for 3 days, and cultured for 5 days. After 5 days, it was confirmed that aging was induced by measuring activities of Lamin B1, which is an aging-related marker, and senescence-associated beta-galactosidase.

The aging-induced skin keratinocytes were pre-treated with 50 ppm of Jangwon No. 3 extract (Example 1), 50 ppm of general green tea extract (Comparative Example 1), or DMSO (dimethyl sulfoxide, control group) for 24 hours, and then were synchronized by culturing for 2 hours in a KBM (keratinocyte basal medium) medium comprising 50% FBS (fetal bovine serum). Thereafter, at 6, 12, 18, and 24 hours, RNA was extracted using an RNeasy mini kit (Qiagen), and after synthesizing cDNA from the extracted RNA using a SuperScript VILO Master Mix (Invitrogen), expression amount changes of Bmal1 (Hs00154147_m, Applied Biosystems, Foster City, CA, USA) were observed by real-time qPCR (a 7500 Fast Real-time PCR System, Thermo Fisher Scientific, Waltham, MA, USA).

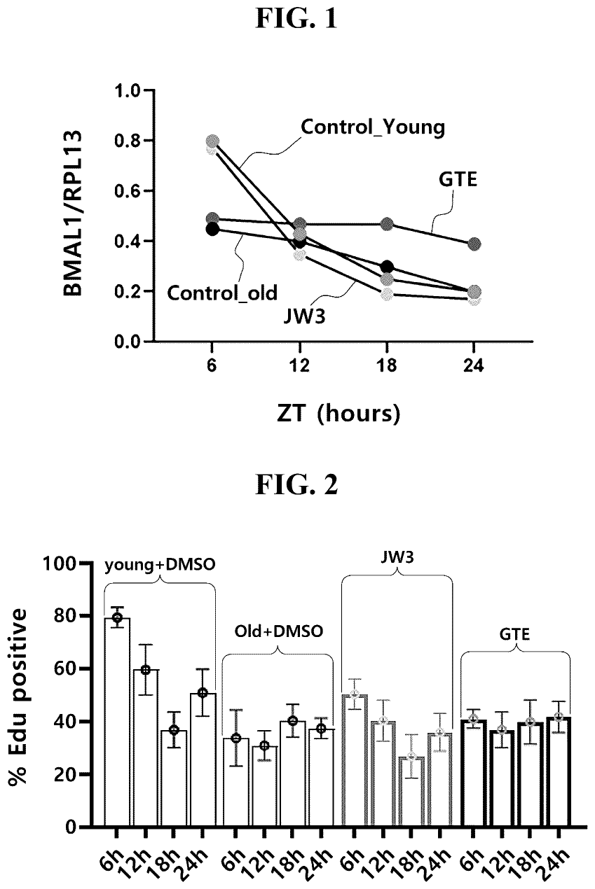

As a result, as shown in FIG. 1, in the aging-induced control group (Control_old), the Bmal1 expression amount is significantly decreased than in cells before aging induction (Control_Young), and almost no change in the Bmal1 expression amount appeared according to a lapse of time even upon treatment of the general green tea extract (Green tea extract (GTE), Comparative Example 1), whereas it can be confirmed that for the Jangwon No. 3 extract (JW3, Example 1) according to one embodiment of the present disclosure, the expression amount was promoted to a level close to skin keratinocytes (Young) in which aging was not induced. This means that the Jangwon No. 3 tea plant extract, which is the active ingredient of the present disclosure, has an effect of promoting expression of a clock gene Bmal1 protein decreased by aging which does not appear in the general green tea extract, and accordingly, circadian rhythm disorders of skin can be prevented or improved.

### [Test Example 3] Verification of Time Dependence Recovery Effect of Cell Growth Cycle

The aging-induced skin keratinocytes prepared in Test Example 2 were pre-treated with 50 ppm of Jangwon No. 3 extract (Example 1) or 50 ppm of general green tea extract (Comparative Example 1) for 24 hours, and then were synchronized by culturing for 2 hours in a keratinocyte basal medium (KBM) medium comprising 50% FBS. Thereafter, a 10 μM EdU (5-ethynyl-2′-deoxyuridine) component was treated, and at 6, 12, 18, and 24 hours, an amount of replicated DNA was measured using Zen software (Carl Zeiss, Jena, Germany) under a fluorescence microscope (Zeiss LSM700 confocal microscope) by attaching a fluorescence probe (Click-iT™ Plus EdU Cell Proliferation Kit for Imaging, Alexa Fluor™ 488 dye, Invitrogen) to the EdU component bound to DNA.

As a result, as shown in FIG. 2, compared with the aging-induced control group (Old_DMSO), almost no cell growth change according to time appeared upon treatment of the general green tea extract (Green tea extract (GTE), Comparative Example 1), whereas it can be confirmed that upon treatment of the Jangwon No. 3 extract (JW3, Example 1) according to one embodiment of the present disclosure, time dependence of a cell growth cycle is recovered, showing a pattern similar to skin cells before aging induction (Young_DMSO). This means that the Jangwon No. 3 tea plant extract, which is the active ingredient of the present disclosure, exhibits a recovery effect of the time dependence of the cell growth cycle which does not appear in the general green tea extract.

### [Test Example 4] Verification of Improvement Effect of Sensitivity to Ultraviolet Rays

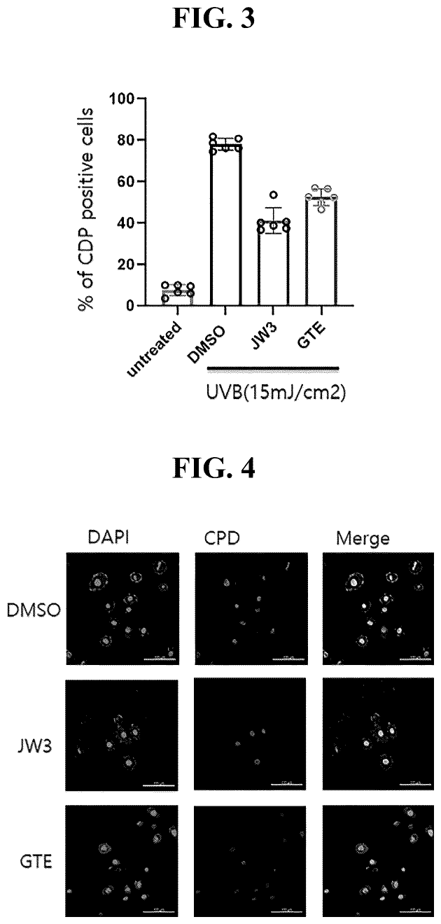

The aging-induced skin keratinocytes prepared in Test Example 2 were pre-treated with 50 ppm of Jangwon No. 3 extract (Example 1) or 50 ppm of general green tea extract (Comparative Example 1) for 24 hours, and then were synchronized by culturing for 2 hours in a keratinocyte basal medium (KBM) medium comprising 50% FBS. After 18 hours, the cells were washed with phosphate-buffered saline (PBS), and in a state where the PBS was added, UVB 15 mJ/cm² was irradiated, and the medium was exchanged with a medium treated with a drug. After a lapse of 1, 4, and 24 hours, an amount of a pyrimidine dimer generated by ultraviolet irradiation was measured and is shown in FIG. 3. That is, at each time, the cells were washed with PBS, then fixed with 3.7% formaldehyde, and treated with 2M HCl to denature DNA. Thereafter, blocking was performed in PBS comprising 20% FBS, and an antibody against the pyrimidine dimer (Anti-cyclobutane pyrimidine dimers (CPDs)) was reacted for 30 minutes. Next, a fluorescence-attached secondary antibody (Goat anti-Rabbit IgG (H+L) Cross-Adsorbed Secondary Antibody, Alexa Fluor™ 594, Invitrogen) was reacted and analyzed using a fluorescence microscope (Zeiss LSM700 confocal microscope) and is shown in FIG. 4.

As a result, as shown in FIG. 3 and FIG. 4, it can be confirmed that an amount of CPD (damaged DNA fragments) is decreased upon treatment of the Jangwon No. 3 extract (JW3, Example 1) according to one embodiment of the present disclosure compared with the general green tea extract (Green tea extract (GTE), Comparative Example 1). This means that when the Jangwon No. 3 tea plant extract, which is the active ingredient of the present disclosure, is compared with the general green tea extract, DNA damaged by ultraviolet rays is significantly decreased, and thus sensitivity due to ultraviolet irradiation is also improved.

### [Test Example 5] Verification of Ultraviolet Resistance Enhancement Effect

To compare an ultraviolet resistance enhancement effect of the extracts of Example 1 and Comparative Example 1, the following experiment was performed.

To an artificial epidermis model (Epiderm™ EPI-201, MatTek Corporation, Ashland, MA, USA), ultraviolet rays 100 mJ/cm² were irradiated, and then 50 ppm of Jangwon No. 3 extract (Example 1) or 50 ppm of general green tea extract (Comparative Example 1) or DMSO (control group) was treated for 2 days, and tissue was fixed with formalin and subjected to hematoxylin & eosin (H&E) staining. Also, the tissue was stained using antibodies against filaggrin (FLG), which is a barrier marker, and Ki67, which is a cell proliferation marker.

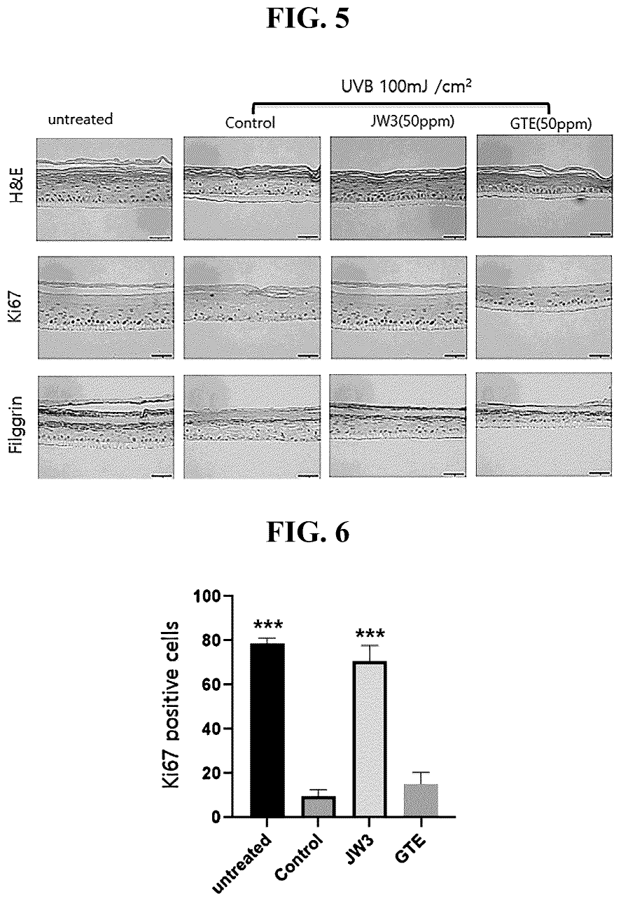

As a result, as shown in FIG. 5, it can be confirmed through dead skin cells and an epidermal structure by the hematoxylin & eosin (H&E) staining that epidermal tissue is damaged by ultraviolet irradiation. When compared with the general green tea extract (Green tea extract (GTE), Comparative Example 1), it can be confirmed through the dead skin cells and the epidermal structure that the one treated with the Jangwon No. 3 extract is recovered to a level close to before skin damage induction (untreated).

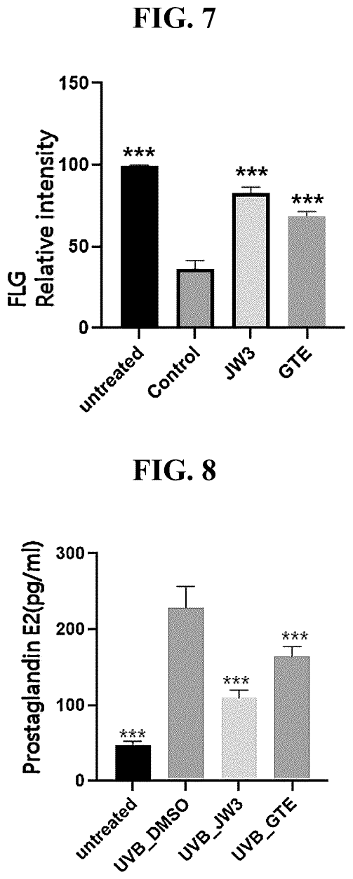

FIG. 6 and FIG. 7 show a comparison of a number of cells stained with Ki67, which is a skin proliferation marker, and an intensity of filaggrin (FLG), which is a barrier marker, respectively, and almost no difference appeared from a control group (control) subjected to ultraviolet irradiation upon treatment of the general green tea extract (Green tea extract (GTE), Comparative Example 1), whereas it can be confirmed that upon treatment of the Jangwon No. 3 extract (JW3, Example 1) according to one embodiment of the present disclosure, among the proliferation marker and the barrier marker, particularly the proliferation marker is recovered to a level close to before skin damage induction (untreated). This means that the Jangwon No. 3 tea plant extract, which is the active ingredient of the present disclosure, does not simply exhibit a protective effect against ultraviolet rays compared with the general green tea extract, but cell proliferation itself is improved by improving sensitivity of cells themselves to ultraviolet rays.

### [Test Example 6] Verification of Erythema Improvement Effect Due to Ultraviolet Rays

To compare an erythema improvement effect due to ultraviolet rays of the extracts of Example 1 and Comparative Example 1, the following experiment was performed.

In Test Example 5, to the artificial epidermis model (Epiderm™ EPI-201, MatTek Corporation, Ashland, MA, USA), ultraviolet rays 100 mJ/cm² were irradiated, and then 50 ppm of Jangwon No. 3 extract (Example 1) or 50 ppm of general green tea extract (Comparative Example 1) or DMSO (control group) was treated for 2 days, and an amount of prostaglandin (PG) E2, which most affects erythema in the medium, was measured using a PGE2 ELISA kit (Prostaglandin E2 Parameter Assay Kit, R&D Systems).

As a result, as shown in FIG. 8, it can be confirmed that a prostaglandin concentration is decreased by 2 times to 3 times or more upon treatment of the Jangwon No. 3 extract (JW3, Example 1) according to one embodiment of the present disclosure compared with the general green tea extract (Green tea extract (GTE), Comparative Example 1). This means that the Jangwon No. 3 tea plant extract, which is the active ingredient of the present disclosure, exhibits a very excellent erythema prevention and improvement effect due to ultraviolet rays.

Formulation Examples of the composition according to one embodiment of the present specification are described below, but application to various other formulations is also possible, and this is not intended to limit the present specification but merely to describe it specifically.

### [Formulation Example 1] Softening Toner (Skin Lotion)

A softening toner was manufactured by a conventional method according to a composition described in the table below.

TABLE 3

| Blending component | Content (wt %) |
|---|---|
| Jangwon No. 3 tea plant extract | 10 |
| Glycerin | 3.0 |
| Butylene glycol | 2.0 |
| Propylene glycol | 2.0 |
| Carboxyvinyl polymer | 0.1 |
| PEG-12 nonylphenyl ether | 0.2 |
| Polysorbate 80 | 0.4 |
| Ethanol | 10.0 |
| Triethanolamine | 0.1 |
| Preservative, colorant, fragrance | q.s. |
| Purified water | balance |

### [Formulation Example 2] Nourishing Toner (Milk Lotion)

A nourishing toner was manufactured by a conventional method according to a composition described in the table below.

TABLE 4

| Blending component | Content (wt %) |
|---|---|
| Jangwon No. 3 tea plant extract | 10 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/Capric triglyceride | 5.0 |
| Squalane | 5.0 |
| Sorbitan sesquioleate | 1.5 |
| Liquid paraffin | 0.5 |
| Cetearyl alcohol | 1.0 |
| Triethanolamine | 0.2 |
| Preservative, colorant, fragrance | q.s. |
| Purified water | balance |

### [Formulation Example 3] Massage Cream

A massage cream was manufactured by a conventional method according to a composition described in the table below.

TABLE 5

| Blending component | Content (wt %) |
|---|---|
| Jangwon No. 3 tea plant extract | 10 |
| Glycerin | 8.0 |
| Butylene glycol | 4.0 |
| Liquid paraffin | 45.0 |
| Beta-glucan | 7.0 |
| Carbomer | 0.1 |
| Caprylic/Capric triglyceride | 3.0 |
| Beeswax | 4.0 |
| Cetearyl glucoside | 1.5 |
| Sorbitan sesquioleate | 0.9 |
| Vaseline | 3.0 |
| Paraffin | 1.5 |
| Preservative, colorant, fragrance | q.s. |
| Purified water | balance |

### [Formulation Example 4] Drink

After mixing 50 mg of Jangwon No. 3 tea plant extract, 10 g of glucose, 0.6 g of citric acid, and 25 g of liquid oligosaccharide, 300 ml of purified water is added and each bottle is filled with 200 ml. After filling the bottles, sterilization was performed at 130° C. for 4 to 5 seconds to manufacture a drink.

### [Formulation Example 5] Caramel Formulation

50 mg of Jangwon No. 3 tea plant extract, 1.8 g of corn syrup, 0.5 g of skim milk, 0.5 g of soy lecithin, 0.6 g of butter, 0.4 g of vegetable hydrogenated oil, 1.4 g of sugar, 0.58 g of margarine, and 20 mg of salt were mixed and caramel-molded.

### [Formulation Example 6] Health Food

TABLE 6

| Component | Content | |
|---|---|---|
| Jangwon No. 3 tea plant extract | 100 | mg |

Vitamin mixture

| | | |
|---|---|---|
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

A composition ratio of the vitamin and mineral mixture was mixed and composed by taking components relatively suitable for health food as an example, but a blending ratio thereof may be arbitrarily modified and implemented, and after mixing the above components according to a conventional health food manufacturing method, granules are manufactured, and it may be used for manufacture of a health food composition according to a conventional method.

### [Formulation Example 7] Health Drink

TABLE 7

| Component | Content | |
|---|---|---|
| Jangwon No. 3 tea plant extract | 10 | mg |
| Citric acid | 1000 | mg |
| Oligosaccharide | 100 | g |
| Plum concentrate | 2 | g |
| Taurine | 1 | g |
| Purified water | balance | |
| Total volume | 900 | ml |

The remaining amount of purified water was added so that a total volume becomes 900 ml as in the table above, and after mixing the above components according to a conventional health drink manufacturing method, and then stirring and heating at 85° C. for about 1 hour, a produced solution is filtered and obtained in a sterilized 2 L container, sealed and sterilized, and then stored under refrigeration to be used for manufacture of a health drink composition.

### Deposit Number

Name of Depositary Institution: Korean Collection for Type Cultures (KCTC), Korea Research Institute of Bioscience and Biotechnology
Deposit number: KCTC12213BP
Deposit date: May 14, 2012

What is claimed is:

1. A method for preventing or improving circadian rhythm disorders of skin, comprising administering a Jangwon No. 3 tea plant extract in an effective amount to a subject in need thereof.

2. The method of claim 1, wherein the extract comprises methylated catechin (epigallocatechin-3-O-(3-O-methyl)-gallate) in 0.5 to 10 wt % relative to a total weight of the extract.

3. The method of claim 2, wherein the extract further comprises one or more selected from the group consisting of gallocatechin, epigallocatechin, catechin, epicatechin, epigallocatechin gallate, gallocatechin gallate, and epicatechin gallate.

4. The method of claim 3, wherein the extract comprises, relative to the total weight of the extract,
0.5 to 10 wt % of methylated catechin;
0.41 to 1 wt % of gallocatechin;
8 to 15 wt % of epigallocatechin;
0.3 to 1 wt % of catechin;
2.1 to 10 wt % of epicatechin;
16 to 30 wt % of epigallocatechin gallate;
0.11 to 0.5 wt % of gallocatechin gallate; and
3 to 15 wt % of epicatechin gallate.

5. The method of claim 1, wherein the Jangwon No. 3 tea plant extract is an aqueous ethanol solution extract of Jangwon No. 3 tea plant leaves.

6. The method of claim 1, wherein the Jangwon No. 3 tea plant extract is comprised in a composition as an active ingredient, and the active ingredient is comprised in 0.0001 to 50 wt % relative to a total weight of the composition.

7. The method of claim 1, wherein the circadian rhythm disorders of skin are generated by biological rhythm disturbance.

8. The method of claim 1, wherein the method is for preventing or improving skin damage due to ultraviolet rays through improvement of circadian rhythm disorders of skin.

9. The method of claim 1, wherein the Jangwon No. 3 tea plant extract is comprised in a composition as an active ingredient, and the composition is a cosmetic composition.

10. The method of claim 1, wherein the Jangwon No. 3 tea plant extract is comprised in a composition as an active ingredient, and the composition is a food composition.
